(19) United States
(12) Patent Application Publication
(10) Pub. No.: US 2011/0160555 A1
(43) Pub. Date: Jun. 30, 2011

(54) Universal Models for Predicting Glucose Concentration in Humans

(76) Inventors: Jacques Reifman, New Market, MD (US); Adiwinata Gani, Jakarta (ID); Andrei Gribok, Boonsboro, MD (US); Srinivasan Rajaraman, Frederick, MD (US)

(21) Appl. No.: 13/056,655

(22) PCT Filed: Jul. 31, 2009
(86) PCT No.: PCT/US09/52505

§ 371 Date: Jan. 29, 2011

Related U.S. Application Data
(60) Provisional application No. 61/085,295, filed on Jul. 31, 2008.

(51) Int. Cl. A61B 5/145

(52) U.S. Cl. 600/365

(57) ABSTRACT

An embodiment of the invention provides a system for predicting future glucose levels in an individual including a glucose measuring device for generating glucose signals representing glucose levels obtained from the individual at fixed time intervals and an analyzer. The analyzer uses a glucose prediction function that is portable between individuals irrespective of health of the individuals. The glucose prediction function includes model coefficients that are invariant between the individuals. The glucose prediction function outputs the future glucose levels by weighing the previous glucose signals obtained from the individual by the model coefficients.

### I. FIELD OF THE INVENTION

The present invention is in the field of methodologies, systems, computer program products, and universal models for predicting glucose concentration in humans.

### II. BACKGROUND OF THE INVENTION

Within this application several publications are referenced by Arabic numerals within brackets. Full citations for these, and other, publications may be found at the end of the specification immediately preceding the claims. The disclosures of all these publications in their entireties are hereby expressly incorporated by reference into the present application for the purposes of indicating the background of the present invention and illustrating the state of the art. If however there are any conflicts between this disclosure and text incorporated by reference, then statements made in this document control and supersede the incorporated teachings.

Minimally invasive continuous glucose monitoring (CGM) devices are instruments utilized to measure and record a patient's glycemic state as frequently as every minute [1]. This information can be utilized to alter or improve the patient's lifestyle, to tighten their glycemic control, or to adjust therapy. These frequent measurements can also be used by data-driven models to forecast future values of subcutaneous glucose concentration and avoid undesired hypoglycemic or hyperglycemic episodes [1]-[4].

In contrast to intermittent measurements, CGM devices collect information frequently such that consecutive measurements retain a large degree of temporal correlation. This correlation is exploited by data-driven models to infer future values as a function of previous measurements [2]-[4]. However, because of the availability of glucose signals at high sampling rates, developers of data-driven models often implicitly assume that the models need to be tuned for a specific individual, thus increasing the burden of model development and reducing their practical applicability. For example, Sparacino et al. [3] uses an autoregressive (AR) model of order one, AR(1), which continuously adapts the model coefficients to the monitored individual to predict future glucose concentrations up to 30 minutes from the time of prediction. Although such a model can produce acceptable predictions, it needs to be continuously adapted for every individual. Additionally, in spite of the adaptive nature of the model, it introduces a significant delay between predicted and measured values. This delay is caused by the low order of the AR model, because a single AR model coefficient is not sufficient to capture the temporal variations of the time-series glucose signal. In another example, Dua et al. [4] employs a Kalman filter to predict future blood glucose levels by continuously adjusting parameters of a first-principles model. Although the first-principle model is significantly more flexible than the AR(1) model of Sparacino et al., the continuous adaptation also makes the Dua et al. model individual specific.

### III. SUMMARY OF THE INVENTION

At least one embodiment of the invention provides a universal, data-driven model developed based on glucose data from one diabetic subject, which is subsequently applied to predict subcutaneous glucose concentrations of other subjects, even those with different types of diabetes. Three separate studies, each utilizing a different CGM device, were used to verify the model's universality. Two out of the three studies involved subjects with type 1 diabetes and the other study was for type 2 diabetes. The subcutaneous glucose concentration data are filtered (i.e., smoothed) by imposing constraints on their rate of change. Using the filtered data, data-driven autoregressive (AR) models of order 30 are developed and utilized to make short-term, 30-minute-ahead glucose-concentration predictions. Same-subject model predictions are utilized as a reference for comparisons against cross-subject and cross-study model predictions, which are evaluated using the root mean squared error (RMSE). For each studied subject, the average cross-subject and cross-study RMSEs of the predictions are small and indistinguishable from those obtained with the same-subject models. In addition, the predictive capability of the models is not affected by diabetes type, subject age, CGM device, and inter-individual differences. Thus, a stable, universal glucose models is developed that captures the invariant correlations in time-series signals of diabetic patients.

An embodiment of the invention provides a method for predicting at least one future glucose level in an individual. The method receives glucose signals from a glucose measuring device, wherein the glucose signals represent glucose levels obtained from an individual at fixed time intervals. The glucose signals are converted into numerical values representing the glucose levels obtained from the individual. The glucose signals and/or numerical values are stored in a memory unit housed in the glucose measuring device. In another embodiment, the memory unit is external to the glucose measuring device.

The method predicts one or more future glucose levels of the individual by weighing the glucose signals by model coefficients of a glucose prediction function. Weighing the previous glucose signals of the individual by the model coefficients reduces a time lag of the predicted future glucose levels. In at least one embodiment, the predicting of the future glucose level is performed with a processor (or programmable data processing apparatus) having code to perform calculations of the glucose prediction function. The glucose prediction function is a universal autoregressive model that is portable between individuals irrespective of health of the individuals. The health of the individual includes a diabetes type of the individual, age of the individual, and/or whether the individual is hospitalized. Moreover, the model coefficients are invariant between the individuals irrespective of the type of the glucose measuring device utilized to measure the glucose signals.

In addition, the method displays the predicted future glucose levels on a display and generates an alert when the future glucose level of the individual exceeds an upper glucose threshold and/or falls below a lower glucose threshold.

A method according to another embodiment of the invention obtains first glucose measurements (i.e., training data) via a glucose monitoring device. Current glucose levels are monitored at fixed time intervals in a plurality of individuals having type I and type II diabetes (i.e., test subjects). A programmed processor uses a portion of the first glucose measurements to train a glucose prediction function that is portable between individuals. The training of the glucose prediction function is independent of the type of glucose measurement device utilized to obtain the first glucose measurements, the ages of the individuals, and whether the individuals are hospitalized. The training creates model coefficients that are invariant between the individuals.

The method obtains second glucose measurements from the individual using the type of glucose monitoring device utilized to obtain the first glucose measurements, or using a type of glucose monitoring device that is different from the type of glucose monitoring device used to obtain the first glucose measurements. The glucose prediction function is used to predict future glucose levels in the individual. The predicted glucose levels represent glucose levels at least 5 minutes into the future, i.e., 5 minutes from the time that the second glucose measurement is obtained from the individual. Specifically, the model coefficients of the glucose prediction function are multiplied by the second glucose measurements obtained from the individual. Because the model coefficients are invariant between individuals, the predictions are independent of the type of glucose measurement device utilized to obtain the first and second glucose measurement. The predictions are also independent of the diabetes type of the individual, the age of the individual, and whether the individual is hospitalized. The glucose prediction function reduces a time lag of the future glucose levels.

Another embodiment of the invention provides a system for predicting future glucose levels in an individual. A glucose measuring device generates glucose signals representing glucose levels obtained from the individual at fixed time intervals. In at least one embodiment, a memory unit is housed in the glucose measuring device for storing the glucose signals.

A programmed processor housed within the glucose measuring device converts the glucose signals into numerical values representing the glucose levels obtained from the individual. The processor is programmed with a glucose prediction function that is portable between individuals irrespective of health of the individuals. The health of the individual includes the age of the individual, the diabetes type of the individual, and whether the individual is hospitalized. In at least one embodiment of the invention, the glucose prediction function is a universal autoregressive model.

The glucose prediction function includes model coefficients that are invariant between the individuals irrespective of the type of the glucose measuring device utilized to measure the glucose signals. The processor selects the model coefficients based on the sampling rate of glucose measuring device utilized to obtain previous glucose signals from the individual. The glucose prediction function outputs the future glucose levels by weighing the previous glucose signals obtained from the individual by the model coefficients.

The system further includes a display connected to the processor for displaying the future glucose levels. A threshold detector is also provided for generating an alert when a future glucose level of the individual exceeds an upper glucose threshold and/or falls below a lower glucose threshold.

A system according to yet another embodiment of the invention includes one or more glucose measuring devices for measuring current glucose levels in humans. One or more first types of glucose measuring devices are utilized to measure glucose levels from individuals (i.e, test subjects) at fixed time intervals (first output). A second type of glucose measuring device is utilized to measure glucose levels from the individual (second output). In at least one embodiment, the second type of glucose measuring device is different from the first types of glucose measuring devices.

The individuals from which the first output is obtained include individuals having type I and type II diabetes, individuals that are hospitalized, and individuals that are not hospitalized. The individuals range in age from 3 years old to 70 years old. In at least one embodiment of the invention, the average age of the individuals is different from the age of the individual (from which the second output is obtained).

A processor trains a glucose prediction function using the first output from the glucose measuring device. The glucose prediction function is a universal autoregressive model that is portable between individuals. The glucose prediction function includes model coefficients that are invariant between individuals.

In another embodiment, an analyzer uses the trained glucose prediction function and current output from the glucose measuring device to predict the future glucose levels in the individual. The predicted glucose levels represent glucose levels at least 5 minutes into the future, i.e., 5 minutes from the time that the second glucose measurement is obtained from the individual. Because the model coefficients are invariant between individuals, the glucose prediction function predicts the future glucose levels independent of the age of the individual, the diabetes type of the individual, and whether the individual is hospitalized.

### IV. BRIEF DESCRIPTION OF THE DRAWINGS

The present invention is described with reference to the accompanying drawings. In the drawings, like reference numbers indicate identical or functionally similar elements.

FIG. 1A illustrates a flow diagram for a method of predicting at least one future glucose level in an individual according to an embodiment of the invention;

FIG. 1B illustrates a flow diagram for a method of predicting at least one future glucose level in an individual according to another embodiment of the invention;

FIG. 2A illustrates a system for predicting at least one future glucose level in an individual according to an embodiment of the invention;

FIG. 2B illustrates a system for predicting at least one future glucose level in an individual according to another embodiment of the invention;

FIG. 3 is a table illustrating three independent studies using three different CGM systems;

FIG. 4 illustrates a graph including the values of the AR model coefficients according to an embodiment of the invention;

FIG. 5A is a table illustrating the values of thirty model coefficients according to an embodiment of the invention;

FIG. 5B is a table illustrating the values of thirty model coefficients according to another embodiment of the invention;

FIG. 6 is a table illustrating root mean squared errors (RMSEs) and prediction time lags for iSense study subjects tested using different models from three validation scenarios;

FIG. 7 is a table illustrating root mean squared errors (RMSEs) and prediction time lags for Guardian RT study subjects tested using different models from three validation scenarios;

FIG. 8 is a table illustrating root mean squared errors (RMSEs) and prediction time lags for DexCom study subjects tested using different models from three validation scenarios;

FIG. 9A illustrates a graph including raw and smoothed glucose signals;

FIG. 9B illustrates a graph including 30-minute-ahead predictions for four different models;

FIG. 10 illustrates a graph including a error grid analysis scatter plot for the four model predictions in FIG. 9B;

FIG. 11 is a table illustrating the cumulative number of hypo- and hyperglycemic episodes and related statistics (averaged over the corresponding subjects) for the raw, smoothed, and predicted data for each of the three studies; and

FIG. 12 illustrates a graph including the power spectrum density profiles for three studies.

### V. DETAILED DESCRIPTION OF THE DRAWINGS

Exemplary, non-limiting, embodiments of the present invention are discussed in detail below. While specific configurations are discussed to provide a clear understanding, it should be understood that the disclosed configurations are provided for illustration purposes only. A person of ordinary skill in the art will recognize that other configurations may be used without departing from the spirit and scope of the invention.

An embodiment of the invention utilizes similarities in the short-term (30-minute or less) dynamics of glucose regulation in different diabetic individuals to develop a single, universal autoregressive (AR) model for predicting future glucose levels across different patients. Data are collected from three different studies, involving subjects with both type 1 and 2 diabetes and using three different continuous glucose monitoring (CGM) (or glucose monitoring device) devices: iSense (iSense Corporation, Wilsonville, Oreg.), Guardian RT (Medtronic Inc., Northridge, Calif.), and DexCom (DexCom Inc., San Diego, Calif.). Data-driven AR models of a fixed order are developed for each subject; and, the AR models are tested on data from other subjects from the same and from different studies. The RMSE and prediction time lag are used as metrics to quantify the models' performance; and, the resulting AR coefficients from the different models developed for each subject are compared.

The developed AR models (i.e., the AR model coefficients) are not significantly dependent on a given individual, diabetes type, age, or CGM device. Thus, universal, individual-independent predictive models are developed, which reduces the burden of model development as one model can be used to predict future glucose levels in any individual using any CGM device. Such predictive models are utilized together with CGM devices for proactive regulatory therapy.

An embodiment of the invention provides a system for predicting future glucose levels in an individual. The system includes a glucose monitoring device for obtaining time-series data representing glucose levels measured at fixed time intervals from an individual patient. The time-series data is input into a universal AR model having a plurality of model coefficients. As described more fully below, the model coefficients are invariant among patients (i.e., patient/individual independent). In predicting future glucose levels, the model coefficients weight the importance of the previously measured glucose levels (e.g., a more recent measurement may be more important than an older measurement). Thus, each of the measured glucose levels input from the glucose monitoring device is multiplied by a respective model coefficient of the AR model. The models of the embodiments herein use the invariant model coefficients to develop a universal AR model that is portable from individual-to-individual.

The invention in at least one embodiment provides a prediction of a future glucose level. This embodiment uses a desired prediction horizon time for determining the number of times the model is used to process a sliding window of predicted and real glucose levels that advances one sample period per iteration. Each advance removes the oldest glucose level and slides the remaining glucose levels to the next coefficient.

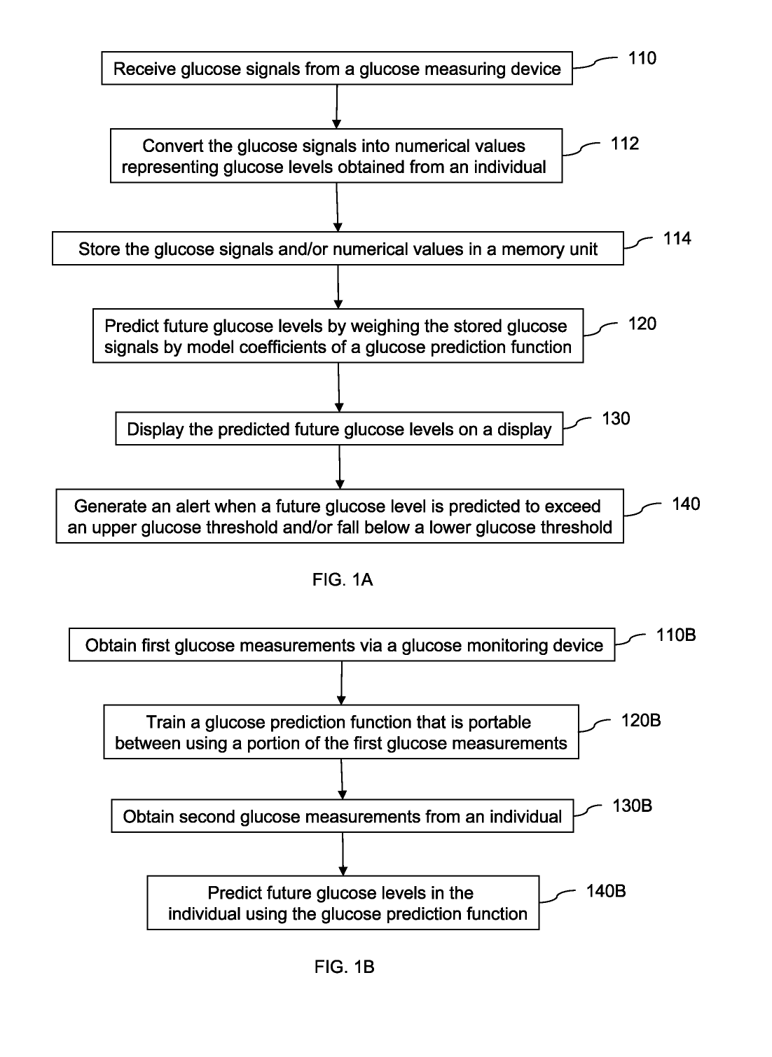

FIG. 1A is a flow diagram illustrating a method for predicting at least one future glucose level in an individual according to an embodiment of the invention. The method receives glucose signals from a glucose measuring device, wherein the glucose signals represent glucose levels obtained from the individual at fixed time intervals (110). For example, in order to predict glucose levels of an individual 30 minutes into the future, glucose levels will need to have been measured for the individual for 30 sampling periods and a number of prediction iterations of the model will be required (e.g., 7 iterations if 5-minute sampling and 31 iterations if 1 minute sampling). The glucose signals are converted into numerical values representing the glucose levels obtained from the individual (112). The glucose signals and/or numerical values are stored in a memory unit housed in the glucose measuring device (114). In another embodiment, the memory unit is external to the glucose measuring device.

The method predicts the individual's future glucose levels by weighing the stored glucose signals by model coefficients of a glucose prediction function (120). The predicting of the future glucose levels is performed with a processor having code to perform calculations of the glucose prediction function.

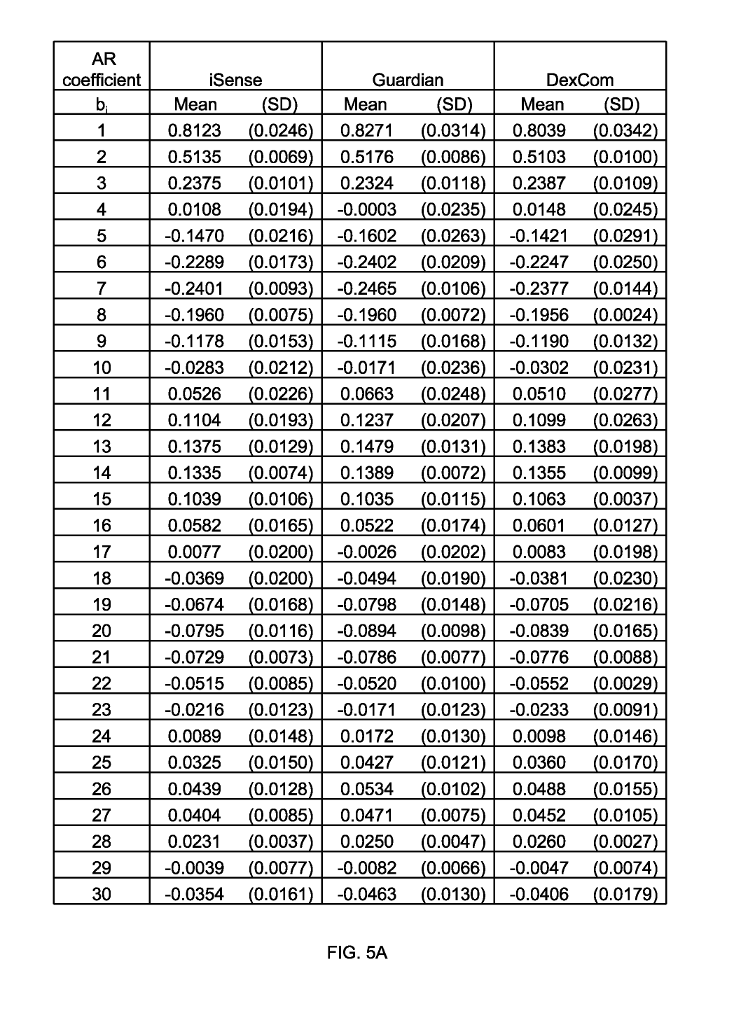

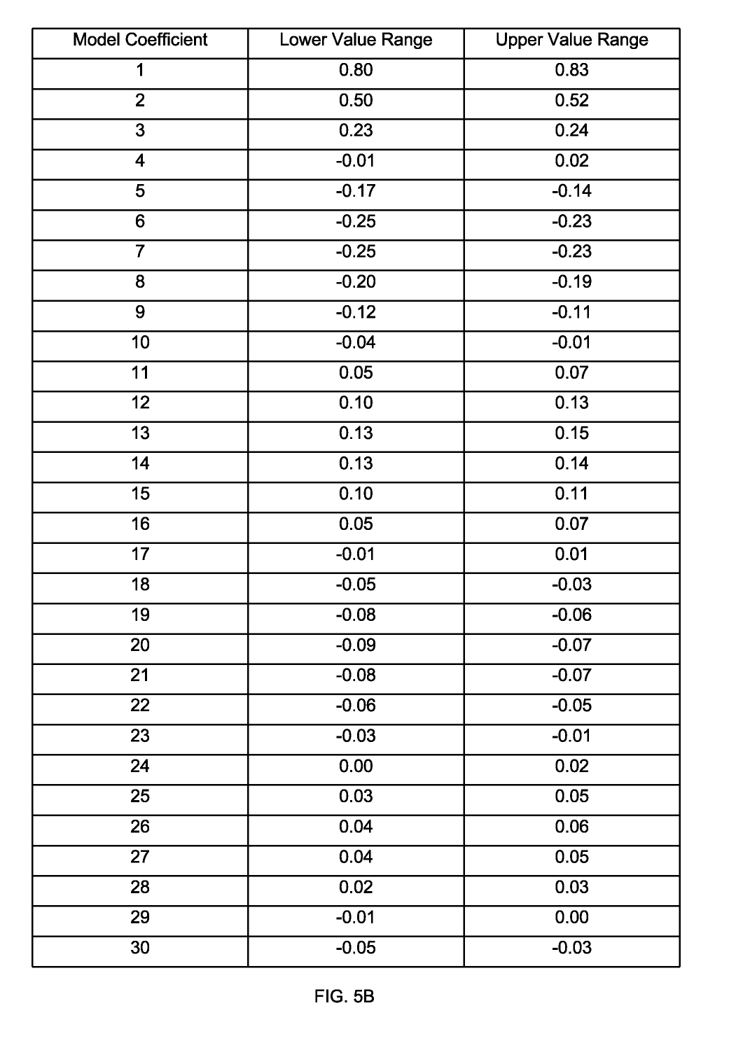

The glucose prediction function is a universal autoregressive model that is portable between individuals irrespective of health of the individuals. The health of the individual includes a diabetes type of the individual, age of the individual, and/or whether the individual is hospitalized. As described more fully below, the glucose prediction function in at least one embodiment is trained using test subjects that include children, adults, and the elderly having type I diabetes and type II diabetes. Moreover, the glucose levels of the test subjects were obtained using three different types of glucose measuring devices. Thus, the model coefficients of the glucose prediction function are invariant between the individuals irrespective of the type of the glucose measuring device utilized to measure the glucose signals. FIG. 5B is a table illustrating the ranges for each of the thirty model coefficients according to at least one embodiment of the invention.

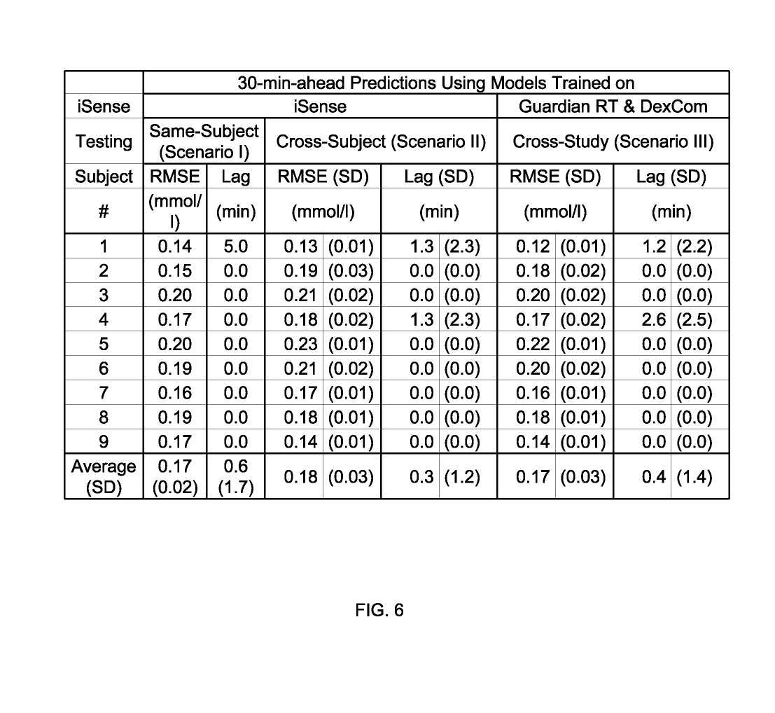

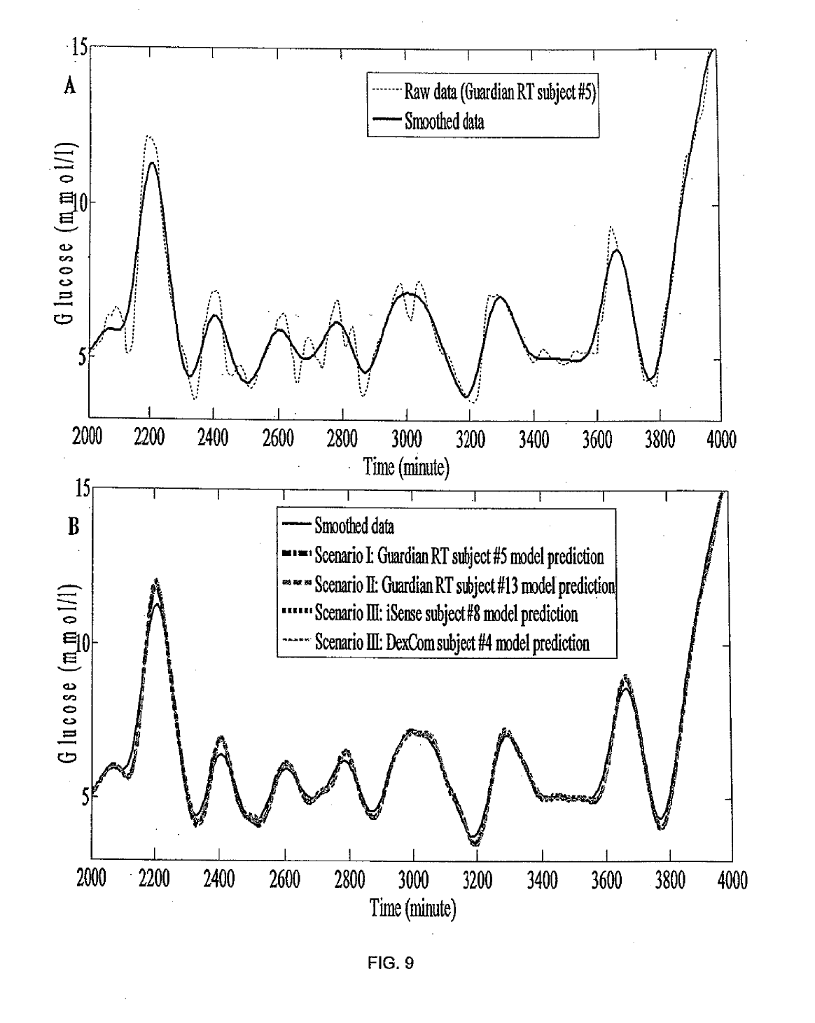

FIG. 9B illustrates future glucose levels predicted by glucose prediction functions according to an embodiment of the invention. The tightness of the data points illustrate that the weighing of the previous glucose signals of the individual by the model coefficients reduces a time lag of the predicted future glucose levels (see also FIGS. 6-8 for actual time lags for 34 glucose prediction functions developed using training data from 34 test subjects).

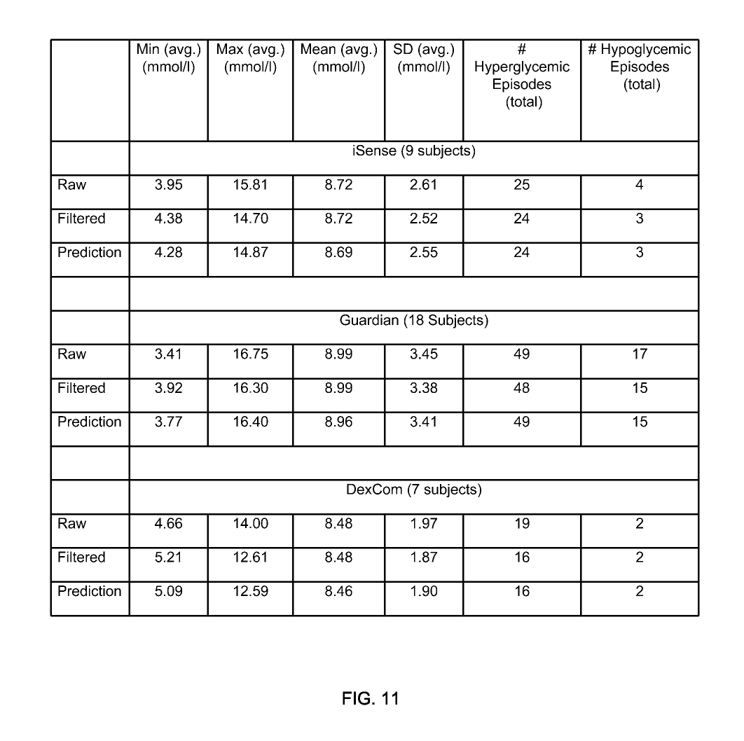

In addition, the method displays the predicted future glucose levels on a display (130) and generates an alert (or other notification) when a future glucose level is predicted to exceed an upper glucose threshold and/or fall below a lower glucose threshold (140). As such, the method in at least one embodiment can be used to avoid hypoglycemic or hyperglycemic episodes. The predicted future glucose levels can be used to alter or improve the patient's lifestyle, to tighten their glycemic control, or to adjust therapy in a proactive manner before an episode occurs. As described more fully below, FIG. 11 is a table illustrating the cumulative number of hypo- and hyperglycemic episodes for the raw (i.e., actual) and predicted data for each of the iSense, Guardian RT, and DexCom studies. The glucose prediction functions correctly predicted 89 out of 93 hyperglycemic episodes (column 6) and 20 out of 23 hypoglycemic episodes (column 7).

FIG. 1B is a flow diagram illustrating a method for training a model and then using the model to predict at least one future glucose level in an individual according to another embodiment of the invention. First glucose measurements (i.e., training data) are obtained via a glucose monitoring device (110B). Current glucose levels are monitored at fixed time intervals in a plurality of individuals having type I and type II diabetes (i.e., test subjects). FIG. 3 illustrates individuals from three separate studies utilized to obtain the first glucose measurements, their diabetes type, sampling interval, and collection time.

A processor uses a portion of the first glucose measurements to train a glucose prediction function that is portable between individuals (120B). In at least one embodiment of the invention, the glucose prediction function is a universal autoregressive model. The training of the glucose prediction function is independent of the type of glucose measurement device utilized to obtain the first glucose measurements, the ages of the individuals, and whether the individuals are hospitalized. As described below in connection with a model training example, the glucose prediction function is trained using test subjects that included children, adults, and the elderly having type I diabetes and type II diabetes.

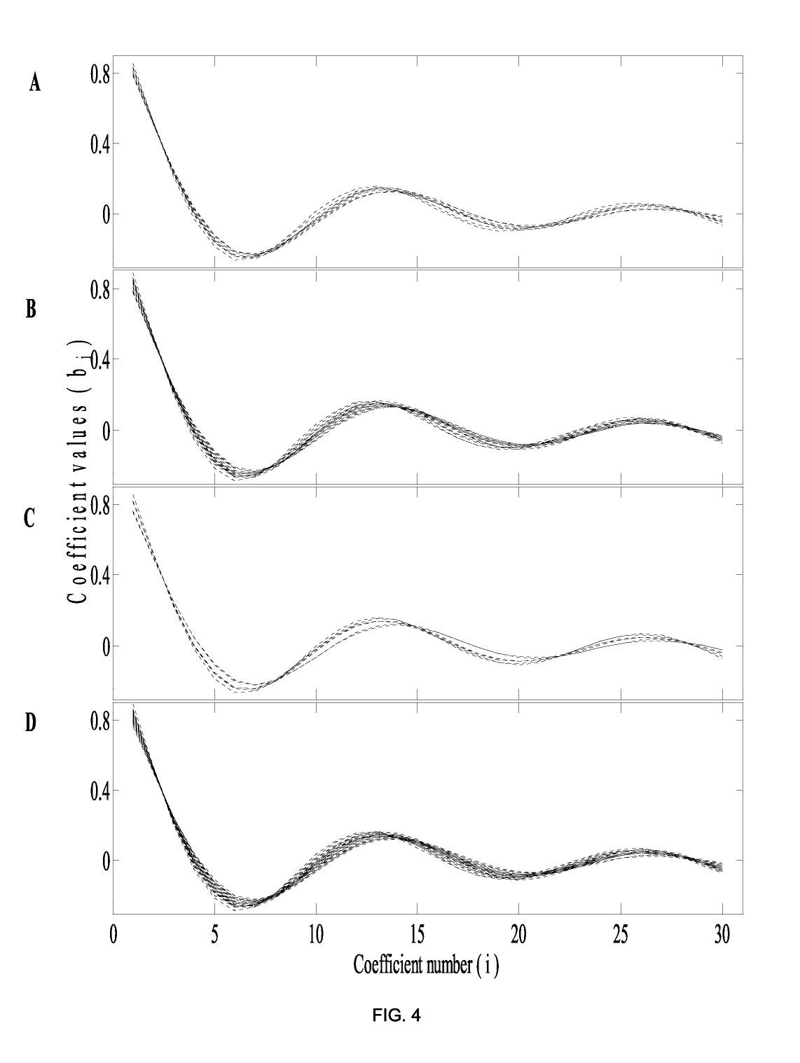

The training creates model coefficients that are invariant between the individuals. As described more fully below in connection with development of example coefficients for a 5-minute sampling period, FIG. 4A illustrates the model coefficients from the first study (iSense); FIG. 4B illustrates the model coefficients from the second study (Guardian RT); FIG. 4C illustrates the model coefficients from the third study (DexCom); and FIG. 4D illustrates the combined model coefficients from the three studies. The tightness in the data points illustrates the invariance of the model coefficients of the 34 test subjects.

The method obtains second glucose measurements from the individual (130B). The second glucose measurements may be obtained using the type of glucose monitoring device utilized to obtain the first glucose measurements, or using a type of glucose monitoring device that is different from the glucose monitoring device used to obtain the first glucose measurements for training.

The glucose prediction function is used to predict future glucose levels in the individual (140B). The predicted glucose levels represent glucose levels at least 5 minutes into the future, i.e., 5 minutes from the time that the second glucose measurement is obtained from the individual. Specifically, the model coefficients of the glucose prediction function are multiplied by the second glucose measurements obtained from the individual. As described below, for example, for a glucose prediction function of order 30 and a 5-minute sampling interval, the most recently measured glucose level \tilde{y}ₙ₋₁ obtained 5 minutes ago is weighed by the first model coefficient bₙ₋₁. Because the model coefficients are invariant between individuals, the predictions are independent of the type of glucose measurement device utilized to obtain the first and second glucose measurement. The predictions are also independent of the diabetes type of the individual, the age of the individual, and whether the individual is hospitalized.

The glucose prediction function reduces a time lag of the future glucose levels. FIG. 9B illustrates future glucose levels predicted by glucose prediction functions according to an embodiment of the invention. The tightness of the data points illustrate minimal time lag of the predicted future glucose levels (see also FIGS. 6-8 for actual time lags for 34 glucose prediction functions developed using training data from 34 test subjects).

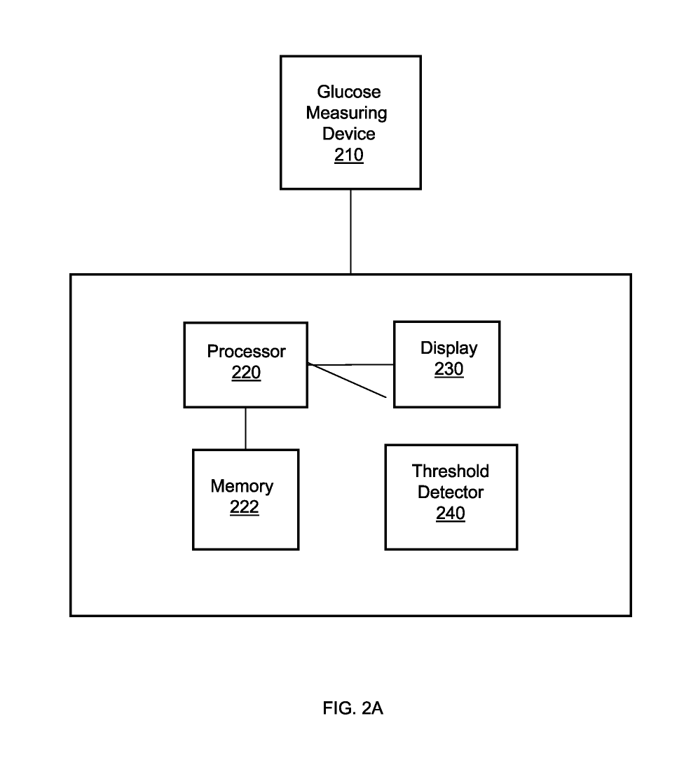

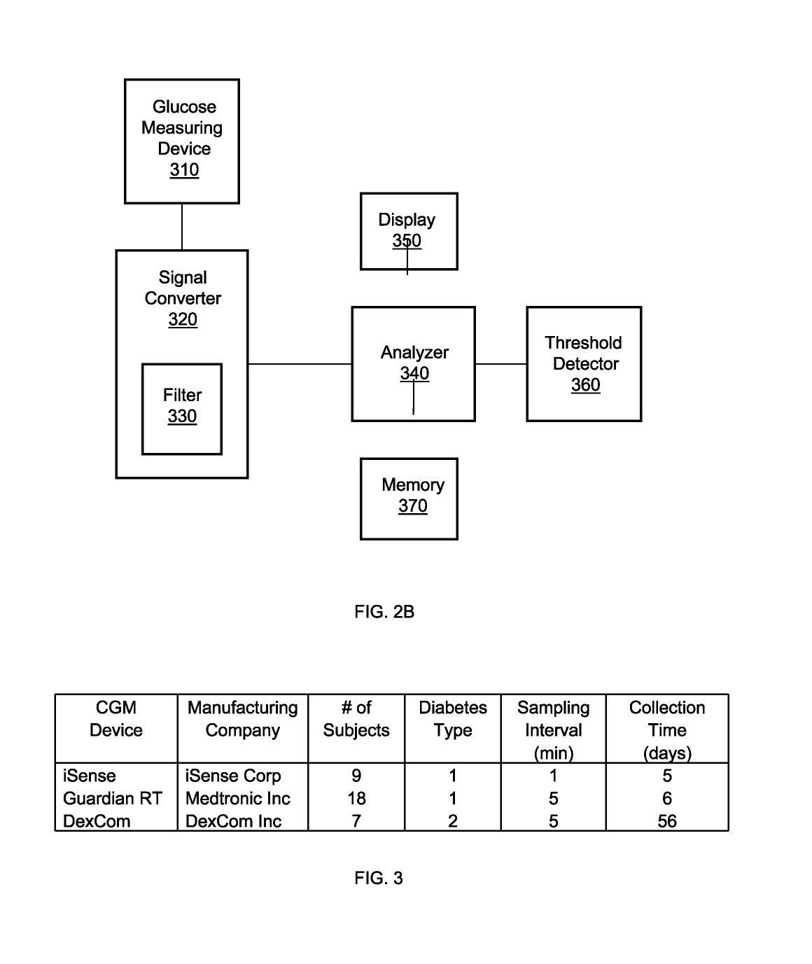

FIG. 2A illustrates a system 200 for predicting at least one future glucose level in an individual according to an embodiment of the invention. A glucose measuring device 210 generates glucose signals representing glucose levels obtained from the individual at fixed time intervals. For example, to predict future glucose levels of the individual, glucose levels are measured from the individual for at least 30 samples, for example, every 5 minutes for 150 minutes or every 2 minutes for 60 minutes.

A processor 220 converts the glucose signals from the glucose measuring device 210 into numerical values representing the glucose levels obtained from the individual. In at least one embodiment, a memory unit 222 is housed in the processor 220 for storing the glucose signals. Although FIG. 2A illustrates that the processor 220 is external to the glucose measuring device 210, the processor 220 is housed within the glucose measuring device 210 in another embodiment of the invention. The processor 220 is programmed to use a glucose prediction function (or predicting means for predicting a future glucose reading) that is portable between individuals irrespective of health of the individuals. The health of the individual includes the age of the individual, the diabetes type of the individual, and whether the individual is hospitalized. In at least one embodiment of the invention, the glucose prediction function is a universal autoregressive model.

The glucose prediction function includes model coefficients that are invariant between the individuals irrespective of the type of the glucose measuring device utilized to measure the glucose signals as described above and below. FIG. 5B is a table illustrating the lower value ranges and upper value ranges of thirty model coefficients according to an embodiment of the invention. In one embodiment, the processor 220 selects the model coefficients based on the sampling rate of glucose measuring device 210 utilized to obtain previous glucose signals from the individual.

The glucose prediction function outputs the future glucose levels by weighing the previous glucose signals obtained from the individual by the model coefficients. As described below, the model coefficients weight the importance of the previously measured glucose levels (e.g., a more recent measurement may be more important than an older measurement). Because the model coefficients describe the correlations in the time-series signal, their absolute values are a function of the sampling frequency of the data used to develop the model. The model coefficients are also dependent on the order of the model. Thus, models having different orders developed on glucose data sampled at different frequencies are expected to yield slightly different model coefficients. The combination of coefficients and order of the model dictate the accuracy of the glucose levels predictions.

The system 200 further includes a display 230 connected to the processor 220 for displaying the future glucose levels. A threshold detector 240 is also provided for generating an alert when a future glucose level of the individual exceeds an upper glucose threshold and/or falls below a lower glucose threshold. As such, the system 200 can be used to avoid hypoglycemic or hyperglycemic episodes. The predicted future glucose levels can be used to alter or improve the patient's lifestyle, to tighten their glycemic control, or to adjust therapy in a proactive manner. The system 200 in an alternative embodiment includes a receiver for communicating with the glucose measuring device 210 when the processor 220 and memory unit 222 are housed in an external unit separate from the glucose measuring device 210. This embodiment also allows the processor 220 to be used with different types of glucose measuring devices 210.

FIG. 2B illustrates a system for predicting future glucose levels of an individual according to an embodiment of the invention. A glucose measuring device 310 generates a series of glucose signals representing glucose levels obtained from the individual at fixed time intervals. A signal converter 320 converts the received glucose signals into numerical values representing the glucose levels obtained from the individual. The signal converter 320 includes computer program instructions loaded onto a processor of a general purpose computer, special purpose computer, application specific integrated circuit (ASIC), or other programmable data processing apparatus, or circuitry. In at least one embodiment of the invention, the signal converter 320 is housed within the glucose measuring device 310. A filter 330 is provided for smoothing the glucose signals to remove high-frequency noise. The filter 330 is in communication with the glucose measuring device 310 and connected to an analyzer 340. In at least one embodiment, the filter 330 is external to the signal converter 320.

The analyzer 340 includes a glucose prediction function that processes the glucose signals (converted or unconverted) in order to predict future glucose levels across a prediction horizon. As described below, the prediction horizon may be input into the analyzer 340 by a user or retrieved from memory 370. In at least one embodiment of the invention, the glucose prediction function is optimized for predicting glucose levels 30 minutes into the future. In one embodiment, the signal converter 320 and the analyzer 340 are co-located in the same device. In another embodiment, the signal converter 320 and the analyzer 340 are integrally connected and present on the same processor or in circuitry.

The glucose prediction function is a universal autoregressive model that is portable between individuals irrespective of health of individuals. The health of the individual includes age of the individual, diabetes type of the individual, and whether the individual is hospitalized. The glucose prediction function includes a plurality of model coefficients that are invariant between individuals irrespective of a type of the glucose measuring device utilized to measure the series of glucose signals. FIG. 5B is a table illustrating the ranges for each of the thirty model coefficients according to at least one embodiment of the invention.

The glucose prediction function outputs the future glucose levels by weighing the current and previous glucose signals obtained from the individual by the model coefficients. As described more fully below, the glucose prediction function outputs a series of future glucose levels by omitting the oldest predicted or actual glucose level used in the last iteration of the glucose prediction function, multiplying a most recent predicted future glucose level by a first model coefficient, and multiplying a next most recent predicted or actual glucose level by a next model coefficient.

As illustrated in FIG. 2B, the system further includes a display 350 connected to the analyzer 340 for displaying the one or more predicted future glucose levels and/or current glucose levels. Examples of displaying multiple future glucose levels are as a curve or a series of numbers. The system in at least one embodiment includes the illustrated threshold detector 360 for generating an alert (or other alarm) when a predicted future glucose level of the individual exceeds an upper glucose threshold or falls below a lower glucose threshold. Examples of alerts include audio, visual, and tactical. In at least one embodiment, the threshold detector 360 is omitted.

Memory 370 is also included in the illustrative embodiment of FIG. 2B. The memory 370 stores the series of glucose signals, the model coefficients, and/or the predicted future glucose levels. For example, the memory 370 stores the glucose signals and predicted future glucose levels in a first in, first out format, such that the glucose prediction function is populated with the most recent glucose levels of the individual (actual or predicted). The memory 370 is in communication with the glucose monitoring device 310 and the analyzer 340.

An embodiment of the invention provides a training system for predicting at least one future glucose level in an individual according to another embodiment of the invention. The system includes one or more glucose measuring devices for measuring current glucose levels in humans. One or more first types of glucose measuring device are utilized to measure glucose levels from individuals (i.e, test subjects) at fixed time intervals (first output). A second type of glucose measuring device is utilized to measure glucose levels from the individual (second output). In at least one embodiment, the second type of glucose measuring device is different from the first types of glucose measuring device.

A glucose prediction function is trained within the processor using the first output from the glucose measuring device. A filter is provided prior to or programmed into the processor for smoothing the first output. As described in more detail later, Tikhonov regularization which yields smoothed signals \tilde{y} by computing \tilde{y}=U_{d}w, where U_{d} denotes the integral operator and w denotes estimates of the glucose signals' first derivatives. The estimates of the derivatives yield excellent data smoothing and do not introduce lag on the smoothed signal relative to the original raw signal.

The glucose prediction function is a universal autoregressive model that is portable between individuals. The glucose prediction function includes model coefficients that are invariant between individuals. FIG. 4A illustrates the thirty model coefficients (x-axis) and the respective values (y-axis) from the first study (iSense). FIG. 4B illustrates the model coefficients from the second study (Guardian RT); FIG. 4C illustrates the model coefficients from the third study (DexCom); and FIG. 4D illustrates the combined model coefficients from the three studies. The tightness in the data points illustrates the invariance in the values of the model coefficients for the 34 test subjects.

The training system includes, for example, a processor or an analyzer that uses the glucose prediction function and second output from the glucose measuring device to predict the future glucose levels in the individual. The predicted glucose levels represent glucose levels at least 5 minutes into the future, i.e., 5 minutes from the time that the second glucose measurement is obtained from the individual. Because the model coefficients are invariant between individuals, the glucose prediction function predicts the future glucose levels independent of the age of the individual, the diabetes type of the individual, and whether the individual is hospitalized.

Yet another embodiment of the invention provides a system for predicting future glucose levels, including means for receiving glucose signals from a glucose measuring device (e.g., a processor, an analyzer). The glucose signals represent glucose levels obtained from an individual at fixed time intervals (e.g., glucose measurements taken every 5 minutes or other sampling period). Means for storing the glucose signals is provided (e.g., a memory unit housed in the glucose measuring device). Means for converting the glucose signals into numerical values is also provided (e.g., a processor or analyzer with or without a filter being connected), wherein the numerical values represent the glucose levels obtained from the individual.

The system in at least one embodiment further includes means for predicting future glucose levels of the individual (e.g., an analyzer or a programmed processor including a computer). Specifically, the means for predicting future glucose levels performs a plurality of iterations of a glucose prediction function by iteratively weighing the glucose signals by model coefficients. The glucose prediction function is portable between individuals irrespective of the health of the individuals. Moreover, the model coefficients are invariant between the individuals.

The system includes means for generating an alert (e.g., a threshold detector with an alert feature) is also provided. The alert is generated when a predicted glucose level exceeds an upper glucose threshold and/or falls below a lower glucose threshold. Examples of the alert feature include an audio alarm, a vibration, a screen displaying or flashing an exemplary word notification, and/or other visual cue (e.g., a warning light).

An embodiment of the invention measures glucose levels in an individual at predetermined intervals to provide a moving window sample to be used to predict a future glucose level. The glucose prediction function is represented by

ŷₙ=\tilde{y}ₙ₋₁b₁+\tilde{y}ₙ₋₂b₂+\tilde{y}ₙ₋₃b₃ . . . +\tilde{y}ₙ₋ₘbₘ

where ŷₙ represents predicted glucose levels; \tilde{y}ₙ₋₁ represents a previously observed glucose measurement; and, b₁ represents a model coefficient. The order of the model is represented by m (i.e., 30 in the example embodiment below). Thus, \tilde{y}ₙ₋ₘ represents the oldest observed glucose level used from the time series; and, \tilde{y}ₙ₋₁ represents the last (or most recently) observed glucose level. The moving window sample will be of the last m readings received from the glucose measuring device. Each observed glucose level is then weighed (i.e., multiplied) by a respective model coefficient.

For example, if the current time is 12:00 pm, an AR model of order 30 taking glucose measurements in 5-minute intervals would need the first measurement (\tilde{y}ₙ₋₃₀) at 9:30 am. Twenty-nine other measurements are taken until the most recent measurement (\tilde{y}ₙ₋₁) is taken at 11:55 am. In order to predict a future glucose level at 12:00 pm, the thirty glucose measurements (\tilde{y}ₙ₋₁-\tilde{y}ₙ₋₃₀) are weighed by respective model coefficients (b₁-b₃₀). For instance, the most recent measurement \tilde{y}ₙ₋₁ is multiplied by b₁. In order to predict a future glucose level at 12:05 pm, the model weighs the twenty-nine most recent actual glucose measurements (\tilde{y}ₙ₋₁-\tilde{y}ₙ₋₂₉) by respective model coefficients (b₂-b₃₀) and the predicted future glucose level at 12:00 is weighed by model coefficient b₁. Similarly, to predict a future glucose level at 12:10 pm, the model weighs the twenty-eight most recent actual glucose measurements (\tilde{y}ₙ₋₁-\tilde{y}ₙ₋₂₈) by respective model coefficients (b₃-b₃₀), the predicted future glucose level at 12:00 is weighed by model coefficient b₂, and the predicted future glucose level at 12:05 is weighed by model coefficient b₁.

In another example, if the model (or prediction function) provides a prediction of the glucose level in the future using an order of 30 with a sampling frequency of 5 minutes, the oldest observed glucose level will have been observed 150 minutes earlier (or at time equal 1 minute (i.e., \tilde{y}ₙ₋ₘ) if the current time is the 146^{th} minute of the sampling) is weighed (i.e., multiplied) by model coefficient b₃₀ (i.e., bₙ₋ₘ). The observed glucose level taken 20 minutes ago (\tilde{y}ₙ₋₄) is weighed by model coefficient b₄; and, the observed glucose level taken at 5 minutes (\tilde{y}ₙ₋₁) is weighed by model coefficient b₁.

For a model using an order of 30 with a sampling frequency of 1 minute, the glucose prediction function would become

Y(30)=y(29)b₁+y(28)b₂ . . . +y(0)b₃₀

where y(29) is the measurement taken at time 29 minutes, i.e., 1 minute ago; y(0) is the measurement taken at time 0 minutes, i.e., 30 minutes ago. To predict the glucose level in 60 minutes at Y(60), then 30 iterations of the equation above are required. For example, if the time is 12:30 pm, in order to predict a future glucose level at 1:00 pm using a model of order 30 and a sampling frequency of 1 minute, the model requires predicted glucose values for every minute between 12:30 and 12:59. However, 30 iterations of the equation are required to predict the future glucose value at 12:59. The above is an example of the functional processing performed by the means for predicting or suitably programmed processors, integrated circuits, chips, or computers.

FIG. 3 is a table illustrating three independent studies using three different CGM systems (iSense, Guardian RT, and DexCom). In the iSense study, nine subjects were confined to the investigational site for the entire duration of the study and limited to mild physical activity. Subjects were included if they were between 18 to 70 years of age, had been diagnosed with type 1 diabetes and treated with insulin for at least 12 months, had body mass index <35.0 kg/m², and had glycated hemoglobin (HbA1c)>6.1%. Subjects are excluded if they had acute and severe illness apart from diabetes, clinically significant abnormal electrocardiogram, hematology or biochemistry screening test, or any disease requiring use of anticoagulants. In addition, subjects were excluded if they were pregnant or lactating. Subcutaneous glucose measurements were collected on a minute-by-minute basis for each of the nine subjects for approximately five days with the iSense CGM system. To standardize the sampling rate across studies, the data was downsampled to 5-minute sampling intervals. The 5-minute sampling interval was half the “optimal” sampling interval (10 minutes) recommended in the literature.

The dataset from the Guardian RT study was retrieved from the Diabetes Research in Children Network (DirecNet) Web site, which makes continuous glucose data for six different studies involving children with type 1 diabetes publicly available, along with the corresponding protocols. Data was obtained from the DirecNet study entitled “A Pilot Study to Evaluate the Navigator Continuous Glucose Sensor in the Management of Type 1 Diabetes in Children,” which included 30 subjects. Subjects were included if they were between 3 and 7 years old or between 12 and 18 years old, had been diagnosed with type 1 diabetes for more than one year, had been using an insulin pump, and had HbA1c≦10.0%. Subjects were excluded if they had significant medical disorder, had severe hypoglycemic event resulting in seizure or loss of consciousness in the last month, had used systemic or inhaled corticosteroids in the last month, or had cystic fibrosis. Subjects were provided with the Guardian RT CGM system for home usage, which collected subcutaneous glucose concentration every 5 minutes for six days. 12 out of the 30 subjects were excluded from the training data because they did not possess consecutive 4,000-minute segments (i.e., 800 data points) without data gaps.

The DexCom study investigates the short- and long-term effectiveness and benefits of frequent CGM measurements versus infrequent CGM measurement (e.g., only before each meal and at bedtime, fingerstick blood glucose measurements). Seven subjects are studied, including an on-going investigation from an independent study. Subjects are included if they were older than 18 years of age, had been diagnosed with type 2 diabetes for at least three months and treated with insulin, and had HbAlc between 7% and 12%. Subjects are excluded if they had been taking glucocorticoids, amphetamines, anabolic, or weight-reducing agents. In addition, subjects were excluded if they were pregnant, lactating, or planning to become pregnant. Subjects continued to take all medications that had been prescribed for diabetes and other medical conditions, and followed their usual meal plans and activity schedules. Investigators of the DexCom study did not make any recommendations to the subjects regarding medications, weight, diet, or exercise at any time during the study. Subjects were instructed to contact their primary care provider for all treatment decisions and consultations. Subcutaneous glucose measurements with the DexCom CGM system were collected every 5 minutes for each of the seven subjects for approximately eight weeks on four two-week cycles.

A model was developed for each one of the 34 subjects that predicted their respective glucose concentrations for a future 30-minute period. To develop the models, glucose signals are obtained from one or more CGM devices. The glucose signals represent the glucose levels taken over a 4,000 minute period (i.e., 800 data points with a 5-minute sampling interval) from the 34 subjects. The glucose signals from each subject are filtered (i.e., smoothed) to remove high-frequency noise. The filtering constrains the glucose rate of change such that the first-order time derivative of the glucose signal is consistent with clinically observed values (i.e., ±0.2 mmol 1⁻¹ min⁻¹ (±4 mg d1⁻¹ min⁻¹)), while avoiding the introduction of time lags between the filtered and the original CGM signals.

An embodiment of the invention utilizes the Tikhonov regularization approach, which yields smoothed signals \tilde{y} by computing \tilde{y}=U_{d}w, where U_{d} denotes the integral operator and w denotes estimates of the glucose signals' first derivatives. The estimates of the derivatives yield excellent data smoothing and do not introduce lag on the smoothed signal relative to the original raw signal. Through this approach, the first derivative or the rate of change of glucose in time is chosen to impose smoothness constraints in the glucose signal. In other words, the smoothed glucose signal \tilde{y} varies minimally from one value to another, thereby ensuring regularity in the underlying signal to be estimated.

To estimate the signal's derivatives w, the functional f(w) is minimized, given by

f(w)=∥y−U_{d}w∥²+λ_{d}²∥L_{d}w∥²

where y denotes the N×1 vector of the raw CGM time-series signal, U_{d} denotes the N×N integral operator, w represents the N×1 vector of first-order differences (the rate of change of glucose with time), λ_{d} represents the data regularization parameter, and L_{d} denotes a well-conditioned matrix chosen to impose smoothness constraints on the derivative of the glucose signal.

For a chosen L_{d}, the quality of smoothing in the aforesaid formulation is determined solely by the regularization parameter λ_{d}. When λ_{d}=0, no regularization is performed, resulting in the original raw CGM data y. As λ_{d} increases, the solution w (and hence \tilde{y}) increasingly satisfies the imposed smoothness constraint, resulting, at the same time, in larger deviations from the raw data.

The first half of each subject's filtered data is utilized to develop an AR model. An AR model is a type of linear model that infers a future signal ŷₙ, at time n (n=m+1, N, where N denotes the total number of data samples available for modeling), based on a linear combination of antecedent samples \tilde{y}ₙ₋ᵢ weighted by a fixed set of coefficients bᵢ,

$${\hat{y}}_{n} = {\sum }_{i = 1}^{m} {b}_{i} {\tilde{y}}_{n - i} ,$$

where m denotes the order of the model, i.e., the number of previously observed and filtered glucose concentrations \tilde{y}ₙ₋ᵢ used to predict a future glucose concentration ŷₙ. This fixed set of coefficients bᵢ, i=1, 2, . . . , m, which defines a model of order m, describes the correlations in the signal. The coefficients are calculated by the method of constrained least squares with an added smoothness constraint to insure physiologic plausibility of the obtained coefficients.

Accordingly, each AR coefficient bᵢ reflects the degree of dependency between the corresponding previous sample \tilde{y}ₙ₋ᵢ and the predicted signal ŷₙ, providing a measure of the physiologic association of the time-series glucose data. Training of an AR model generates the coefficients b that best describe the dependencies in the entire time-series \tilde{y}. In the method of constrained least squares, b is estimated so that the functional ∥\tilde{y}−Ub∥² is minimized, where U denotes the design matrix representing previous values of \tilde{y}.

For glucose concentrations to be predictable with AR models, the CGM data possesses “detectable structure” and the dynamics of the time series data is ideally stationary. By definition, a process is considered stationary when the sample mean and variance of the process measurements are constant with respect to time and the autocorrelation function (ACF) is independent of absolute time. Indication of the stationary nature of the underlying process is therefore sought before applying AR models.

To construct stable AR models, AR model coefficients are obtained through regularization. For a stationary process, the sequence of autocorrelation coefficients representing the ACF describes statistical dependencies between two measurements separated by fixed time intervals throughout the recorded observations. To force the AR coefficients to follow the same statistical dependencies of the ACF, a smoothness constraint is imposed on the method of constrained least squares of the coefficients bᵢ, resulting in the regularized least squares functional g(b), given by

g(b)=∥\tilde{y}−Uₘb∥²+λₘ²∥Lₘb∥²

where \tilde{y} denotes the (N−m)×1 vector of smoothed data, Uₘ denotes the (N−m)×m design matrix, b represents the m×1 vector of regularized AR coefficients, λₘ represents the model regularization parameter, and Lₘ denotes a well-conditioned matrix chosen to impose smoothness on the AR coefficients. Accordingly, the minimization of the above formula results in regularized coefficients b.

Similar to the smoothing of the raw data, for a chosen Lₘ, the stability of the AR model in the above formulation is determined solely by the regularization parameter λₘ. When λₘ=0, no regularization is performed. As λₘ increases, the coefficients are constrained, resulting in more stable, regularized AR coefficients.

The optimal values of the regularization parameters, λ_{d} and λₘ, and the order m of the AR model are estimated. The optimum value of λ_{d} is found by minimizing the sum of the RMSE of the smoothed signal (i.e., the RMSE between the raw and the smoothed signal) and the RMSE of the prediction (i.e., the RMSE between the smoothed signal and its predictions). The RMSE of the smoothed signal is a monotonically increasing function of λ_{d} because the smoother the signal, the more it deviates from the original raw data. Conversely, the RMSE of the prediction is a monotonically decreasing function of λ_{d} because the smoother the signal, the more predictable it becomes. Therefore, by obtaining λ_{d} that minimizes the sum of these two RMSEs, a tradeoff between smoothness and predictability is effectively imposed, resulting in signals with good predictability without oversmoothing. λₘ is selected empirically and m through cross validation.

Once the coefficients are calculated, the models are subsequently used for predicting glucose concentrations, where model performance is quantified by computing prediction time lags and RMSEs. The RMSE is defined as the square root of the mean difference between the predicted signal ŷᵢ and the filtered observed signal \tilde{y}ᵢ, i=1, 2, . . . , N,

$$RMSE = \sqrt{\frac{1}{N} {\sum }_{i = 1}^{N} {( {\hat{y}}_{i} - {\tilde{y}}_{i} )}^{2}} ,$$

and the prediction time lag is calculated based on the cross-correlation between the filtered and predicted signals. The lag, characterized by the peak of the cross-correlation function, provides an accurate estimate of the delay in the predictions.

FIG. 4 is a graph illustrating the model coefficients according to an embodiment of the invention. Specifically, FIG. 4 shows the values of the AR model coefficients bᵢ, i=1, 2, . . . , 30, for: (A) the nine iSense subjects; (B) the 18 Guardian RT subjects; (C) the seven DexCom subjects; and (D) the combined 34 subjects for all three studies. Panel D shows that the model coefficients bᵢ, and hence the glucose models, do not vary significantly from subject-to-subject and from study-to-study, i.e., they are independent of the subject's age, diabetes type, and CGM device used to measure the glucose concentration. Thus, the invariant model coefficients illustrated in FIG. 4 demonstrate that the training datasets from the three studies yield universal glucose models that are portable from individual-to-individual.

In other words, model coefficients bᵢ are derived from the training datasets of 34 subjects in three studies; and, because the derived model coefficients do not differ significantly from subject-to-subject (as demonstrated by the tightness and invariance of the line graphs in FIG. 4), universal models are developed that are portable from individual-to-individual. AR models have two parameters: the model coefficients and the measured data points used to predict future data points. A model coefficient weights the importance of a previously measured data point that is utilized to predict a future data point (e.g., a more recent measurement may be more important than an older measurement). To predict future data points, each measured data point is multiplied by a respective model coefficient (i.e., weighed). The measured data points are different for every patient (i.e., patients will have different glucose levels); however, as illustrated in FIG. 4, the model coefficients are invariant among patients (i.e., subject independent). The models of the embodiments herein use the invariant model coefficients to develop a universal AR model that is portable from individual-to-individual.

FIG. 5A is a table illustrating the mean values of thirty model coefficients b, developed from the training datasets of the three studies usable in at least one embodiment of the invention. FIG. 5A also illustrates standard deviation (SD) values between the model coefficients in each study. For instance, nine models are created from the nine subjects in the iSense study. For these nine models, the mean value for coefficient no. 1 (of 30) is 0.8123. The small standard deviation for coefficient no. 1 between the nine models (i.e., 0.0246) demonstrates the similarity of the AR coefficients in the model. The Guardian study creates eighteen models based on the training data of the eighteen subjects. For these eighteen models, the mean value for coefficient no. 2 is 0.5176. The small standard deviation for coefficient no. 2 between the eighteen models (i.e., 0.0086) demonstrates the similarity of the AR coefficients in the model.

FIG. 5A illustrates that the model coefficients, in particular the ones with relatively large values (>0.05), are similar across the three studies and that their differences are, in general, within one standard deviation. For example, the mean values for model coefficient no. 3 are 0.2375, 0.2324, and 0.2387 for the iSense, Guardian, and DexCom studies, respectively. Thus, a universal model is developed from one subject's data and subsequently used to predict another subject's glucose levels across a short prediction horizon. This completely bypasses the need to develop and fine tune the model for other subjects. However, because the model coefficients describe the correlations in the time-series signal, their absolute values are a function of the sampling frequency of the data used to develop the model. The model coefficients are also dependent on the order of the model. Thus, models having different orders developed on glucose data sampled at different frequencies are expected to yield slightly different model coefficients. The combination of coefficients and order of the model dictate how far into the future glucose levels can be predicted. FIG. 5B is a table illustrating the lower value ranges and upper value ranges of thirty model coefficients according to another embodiment of the invention.

The 34 subjects from the three studies are used to validate the model. The first 2,000 minutes of the filtered signals of each subject are used to train the AR models (training dataset) and the next 2,000 minutes are utilized to test the predictions (testing dataset). The three validation scenarios allow for the comparison of model performance on the same testing datasets by applying distinct models derived from different training datasets. FIG. 6 is a table that illustrates the RMSEs and prediction time lags for the nine iSense subjects tested using different models from the three validation scenarios. In validation scenarios II and III, the RMSEs and time lags are averaged values.

Validation scenario I tests the accuracy of the same-subject models (same subject, same CGM device). More specifically, for each of the 34 subjects, a model is trained on each subject's training dataset (i.e., first 2,000 minutes), resulting in 34 different models. For example, the training dataset for iSense subject #1 is used to derive a model for that subject, which is subsequently used to predict that subject's glucose levels. Each model is validated using the testing dataset (i.e., next 2,000 minutes) of that particular subject. For example, the testing dataset for iSense subject #1 (i.e., actual glucose measurements taken) is compared to the predictions for that subject.

Thus, as illustrated in FIG. 6, the average RMSE (for the 30-minute prediction period) between the actual and predicted glucose levels for iSense subject #1 (using the model developed from the training dataset of iSense subject #1) is 0.14 mmol/l. The average time lag is 5.0 minutes.

Validation scenario II tests the accuracy of the cross-subject models (different subjects, same CGM device). For each subject within a given study, the models developed in scenario I for the remaining subjects of that same study are applied to the testing dataset of the subject. For example, each of the models developed for iSense subjects #2-#9 are applied to the testing dataset of iSense subject #1.

As illustrated in FIG. 6, the average RMSE between the actual and predicted glucose levels for iSense subject #1 (using the models developed from the training datasets of iSense subjects #2-#9) for a 30-minute period is 0.13 mmol/l and the average time lag is 1.3 minutes. The standard deviations for RMSE and time lag are 0.01 mmol/l and 2.3 minutes, respectively.

Validation scenario III tests the accuracy of the cross-study models (different subjects, different CGM devices). For each subject within a given study, the models developed in the other two studies are applied to the testing dataset of the subject. For example, the models developed for the eighteen subjects in the Guardian RT study and the seven subjects in the DexCom study are applied to the testing dataset of subject #1 of the iSense study.

As illustrated in FIG. 6, the average RMSE between the actual and predicted glucose levels for iSense subject #1 (using the models developed for the Guardian RT and DexCom subjects) for a 30-minute period is 0.12 mmol/l and the average time lag is 1.2 minutes. The standard deviations for RMSE and time lag are 0.01 mmol/l and 2.2 minutes, respectively.

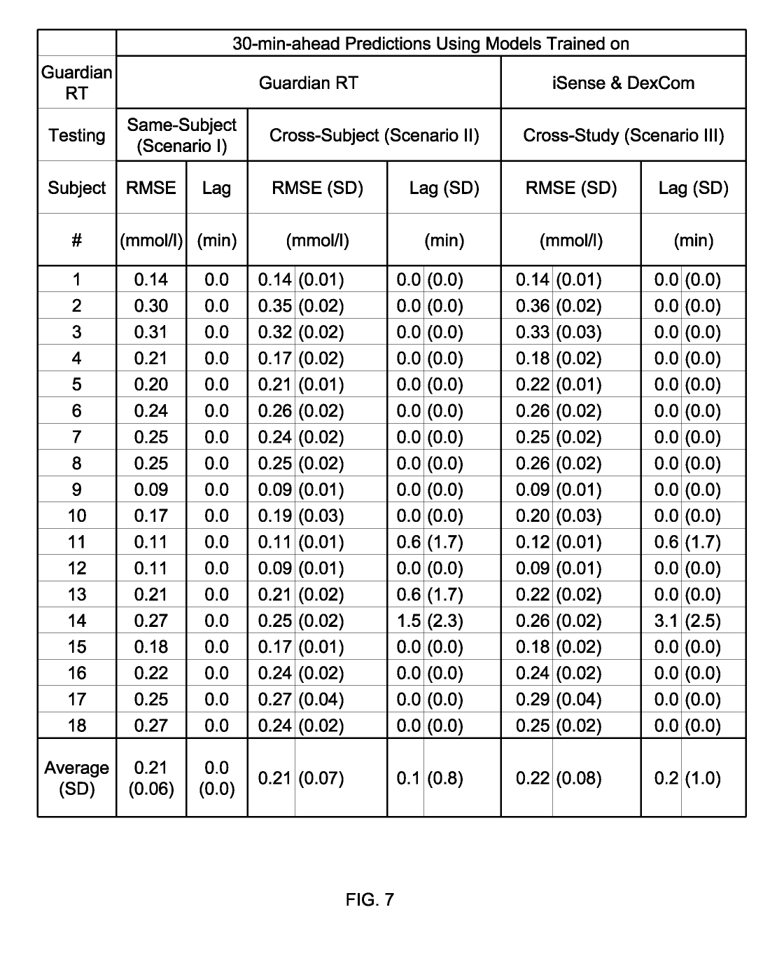

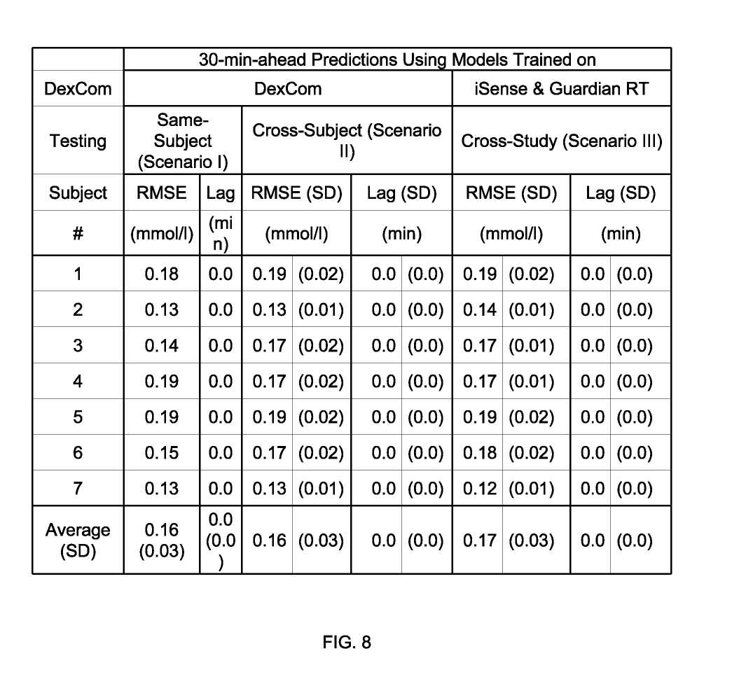

Similar tabulations are shown in FIGS. 7 and 8 for the eighteen Guardian RT subjects and the seven DexCom subjects, respectively. The results in FIGS. 6-8 not only show that the predictive models do not vary significantly (as shown in FIG. 4), but that they also yield very accurate forecasts (i.e., negligible average RMSEs and prediction time lags).

In an example to demonstrate the prediction power of the models, an embodiment of the invention selects a random subject: Guardian RT subject #5. FIG. 9A is a graph illustrating the raw and smoothed glucose signals (measured over the course of the 2,000 minute testing period). FIG. 9A indicates how an algorithm of the filter smoothed the sharp excursions in the raw signal. On average, the filtering process removed about 7% of the signal's energy, which constitutes an acceptable loss. The optimal amount of filtering poses a trade-off between missed and false alarms for hypo- and hyperglycemic episodes. More filtering produces smoother signals and increases the frequency of missed alarms. Conversely, less filtering retains the sharp excursions of the raw signals, increasing the frequency of false alarms.

FIG. 9B is a graph illustrating the 30-minute-ahead predictions for four different models according to an embodiment of the invention, which exemplifies the models' portability in the three validation scenarios. Specifically, for Guardian RT subject #5, FIG. 9B shows the smoothed data (testing dataset from FIG. 9A), the glucose predictions using the model developed for Guardian RT subject #5, the glucose predictions using the model developed for Guardian RT subject #13, the glucose predictions using the model developed for iSense subject #8, and the glucose predictions using the model developed for DexCom subject #4. The prediction results shown in FIG. 9B indicate that the predictions of the Guardian RT subject #5 based on four different models are nearly indistinguishable from one another.

The glucose levels for Guardian RT subject #5 predicted utilizing the model developed using Guardian RT subject #13's training dataset (i.e., the first 2,000 measured glucose data points) illustrate model portability across different subjects within the same study (scenario II). Similarly, the glucose levels for Guardian RT subject #5 predicted utilizing the model developed using iSense subject #8's training dataset and DexCom subject #4's training dataset demonstrate portability across different studies and across different types of diabetes (scenario III). The same-subject predictions (model derived utilizing the training dataset for Guardian RT subject #5) in scenario I serve as a reference for comparison among the different models. Specifically, for validation scenarios I and II, the resulting RMSE's for Guardian RT subject #5 are 0.20 mmol/l and 0.21 mmol/l, respectively. For validation scenario III, using the iSense subject #8 model and DexCom subject #4 model, the resulting RMSE's are 0.22 mmol/l and 0.24 mmol/l, respectively.

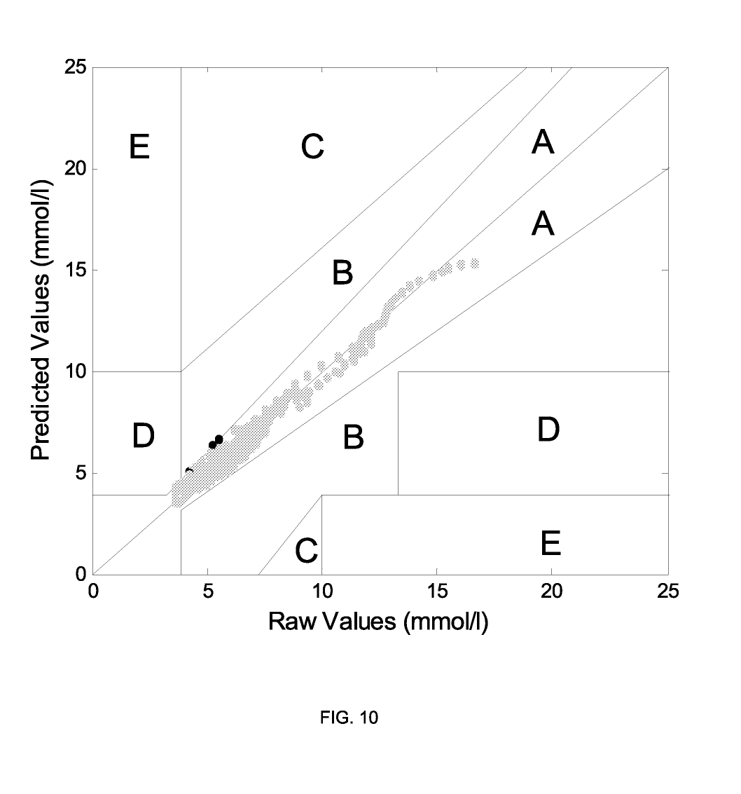

To assess the utility of the glucose predictions using clinically acceptable metrics, a Clarke error grid analysis (EGA) is performed, which maps pairs of sensor-predicted glucose concentrations into five zones, A to E, of varying degrees of accuracy and inaccuracy of glucose estimation. Values in zones A and B are clinically acceptable; values in zone C may result in unnecessary corrections; values in zone D could lead to incorrect treatments and detections; and, values in zone E represent erroneous treatment. FIG. 10 is a graph illustrating the EGA scatter plot for the Guardian RT subject #5 corresponding to the four model predictions in FIG. 9B according to an embodiment of the invention. Each of the 1,600 predictions, 400 for each model, is paired with the corresponding raw glucose concentration in FIG. 9A. Of the 1,600 data points, 1,588 (or 99.25%) lay in zone A; and, 12 data points (or 0.75%) lay in zone B. For the 12 points in zone B, each of the four models contribute three points, and these points correspond to predictions at two time instances, 2150 and 2660 minutes, where the deviations between the raw and the smoothed signals were the largest (see FIG. 9A). These results further demonstrated the equivalent predictive power obtained with the same-subject model, the cross-subject model, and the cross-study model.

The Clarke EGA is also performed for each of the three studies using the same-subject model predictions (scenario I). The composite result of each analysis is plotted on a separate graph (not shown). Of the 3,600 entries (400 data points×9 subjects) for the iSense study, 3,564 points (99.0%) lay in zone A, 35 in zone B, and 1 in zone D. Of the 7,200 entries (400×18) for the Guardian RT study, 7,150 points (99.3%), 32 points, and 18 points lay in zones A, B, and D, respectively. Similarly, of the 2,800 entries of the DexCom study, 2,787 (99.5%), 12, and 1 lay in zones A, B, and D, respectively. These results demonstrated the clinical utility of the predictive models.

To verify that the employed datasets do not correspond to well-treated diabetic patients with glucose levels mostly within the euglycemic range and that the filtering procedure does not over-smooth the raw data, the number of hypo- and hyperglycemic episodes in the raw, smoothed, and predicted data are calculated. A lower threshold of 3.9 mmol/1 (70 mg/dl) and an upper threshold of 10 mmol/1 (180 mg/dl) was adopted; and, an inter-episode separation of at least 30 minutes and a minimum of 30 minutes (seven consecutive data points) outside the euglycemic range were required to count the excursion as a hypo- or hyperglycemic episode. FIG. 11 is a table illustrating the cumulative number of hypo- and hyperglycemic episodes and related statistics (averaged over the corresponding subjects) for the raw, smoothed, and predicted data for each of the three studies. The results confirmed that the subjects did exhibit glucose excursions and that the filtering did not significantly smoothed them out. Overall, the models correctly predicted 89 out of 93 hyperglycemic episodes and 20 out of 23 hypoglycemic episodes.

For instance, for the iSense study, the average minimum glucose levels (in mmol/l) was 3.95, 4.38, and 4.28 for the raw data, smoothed data, and predicted data, respectively. The average maximum glucose levels (in mmol/l) were 15.81, 14.70, and 14.87 for the raw data, smoothed data, and predicted data, respectively. The average mean glucose levels (in mmol/l) were 8.72, 8.72, and 8.69 for the raw data, smoothed data, and predicted data, respectively; and the average standard deviations were 2.61, 2.52, and 2.55 for the raw data, smoothed data, and predicted data, respectively. The total number of hyperglycemic episodes were 25, 24, and 24 for the raw data, smoothed data, and predicted data, respectively; and, the total number of hypoglycemic episodes were 4, 3, and 3 for the raw data, smoothed data, and predicted data, respectively.

The portability properties demonstrated by the models herein are attributed to two factors: the conserved nature of the frequency content in the glucose signal of diabetic patients and the properties of the modeling approach. The dynamics in the blood glucose time-series signal of diabetic patients can be characterized by four distinct frequency ranges. These different frequency ranges characterize different physiologic mechanisms and are best described by the periodicity of their oscillations. The highest frequency range, with periods between 5 and 15 minutes, is generated by pulsatile secretion of insulin. The second highest, ultradian glucose oscillations, corresponds to periods between 60 and 120 minutes. Exogenous inputs, such as meals and insulin, generate oscillations with periods between 150 and 500 minutes; and, finally, circadian oscillations are responsible for the low-frequency range, with periods longer than 700 minutes.

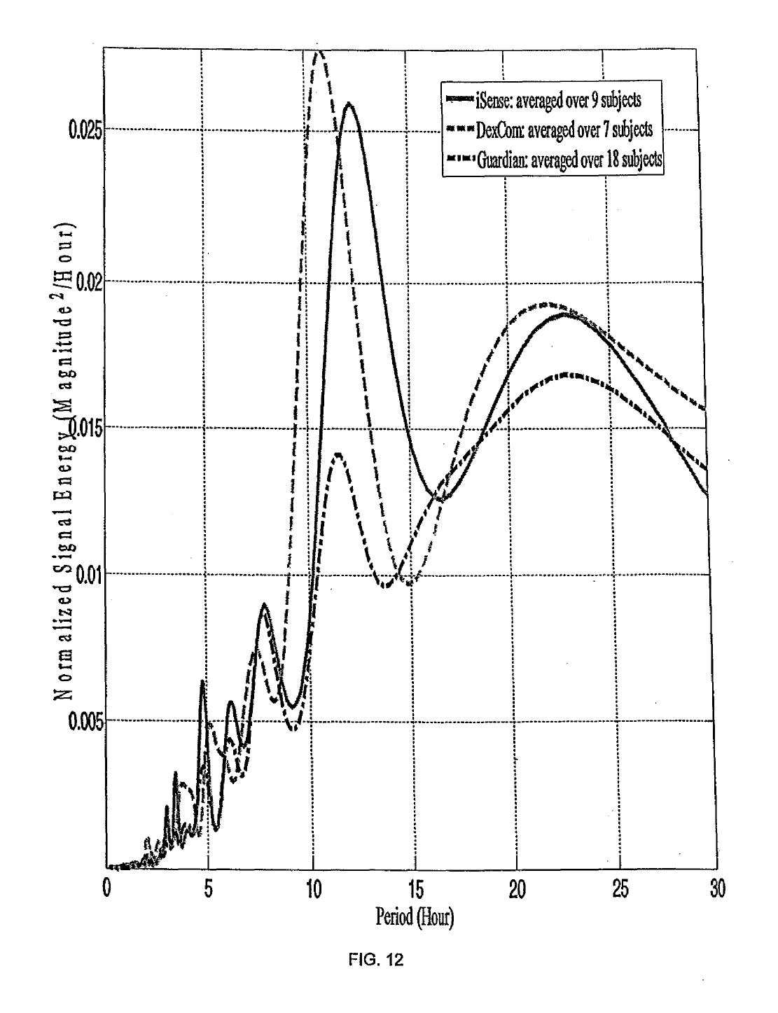

Analysis of the time-series glucose signals of all subjects in the three studies supports these findings and shows that the frequency content in the signals is conserved across subjects. FIG. 12 is a graph illustrating the power spectrum density profiles for each of the three studies, averaged over the subjects in each study. While the amplitudes of the profiles are different for each of the studies, the periodicity (i.e., the location of the peaks on the x-axis) is conserved across the studies. The conservation of biological rhythms, such as the circadian rhythm, across species, or even kingdoms, is a known phenomenon.

This similarity in the frequency content of the glucose signals is exploited by the predictive AR models herein. Periodic signals, like glucose concentration, are characterized by three parameters: amplitude, frequency, and phase of the underlying oscillations. However, a property of AR models is their invariance with respect to a signal's amplitude and phase, and sole dependency on its frequency. The sequence of the AR model coefficients captures and represents the frequency content of a time-series signal. Therefore, the development of the predictive AR models from signals with similar frequency content produced similar (or portable) models, regardless that different time-series signals recorded from different subjects had different amplitudes and initial phases. This invariance of the AR model coefficients to the glucose signal's amplitude and phase affords model portability across subjects with type 1 and type 2 diabetes. Type 1 diabetes patients usually have larger glucose-level variations than type 2 patients. However, if these variations contain the same frequency information, the predictive AR models herein are portable across them. Moreover, because of the frequency-dependent nature of the AR model coefficients, information concerning exogenous inputs, such as meals and exercise, is automatically incorporated into the models if this information is present in the training data.

However, if some of the subjects from the training data are nondiabetic and fasting, the models' portability could be jeopardized because the glucose dynamics are different in this case. This is particularly relevant for the highest-frequency component of the glucose time-series signal, i.e., the shortest periods spanning between 5 and 15 minutes, because while these periods are prominent in nondiabetic, fasting individuals, they are absent in diabetic patients. In diabetic patients, insulin-generating cells responsible for pulsatile secretion of insulin are severely handicapped, essentially eliminating the 5-15 minute periods from the glucose signals. Moreover, the blood-to-interstitial transport acts as a low-pass filter, reducing the high-frequency dynamics in the CGM signals, which are further attenuated by the filtering procedure utilized herein.

The filtering procedure, used to attenuate any remaining high-frequency component in the signal to yield consistent AR coefficients and robust models, does not significantly impact the ability to capture hypo- and hyperglycemic episodes; and hence, the clinical usefulness of at least one embodiment of the invention. FIG. 11 shows that the predictive models herein correctly predicted 96% of the hyperglycemic episodes and 87% of the hypoglycemic episodes present in the three studies.

Another contributing property for the predictive AR model portability relates to the limits imposed on the model coefficients by the constrained least squares method. Besides fitting the AR model to the data, the employed constrained least squares method also limits the curvature (i.e., the norm of the second derivative) of the AR coefficients. This is illustrated in FIG. 4, where the shape of the model coefficients can be loosely described as a dampened sine wave, also reflecting the periodic nature of the glucose signal and that model coefficients that are further apart have weaker correlations than closer ones. This behavior of the AR model coefficients is correct, as the glucose data gradually loses inter-sample correlations as a function of time lag between samples. However, if the curvature constraint is not imposed, unconstrained least squares produces AR model coefficients that exhibit unphysiologic behavior, with model coefficients corresponding to further apart (and less correlated) glucose samples contributing more to the predictions than more correlated, closer ones.

FIG. 7 shows that although the models are portable, their performance, in terms of RMSE, may vary from subject to subject. For example, the RMSE for subject #9 in scenario I is 0.09 mmol/l, whereas for subject #2 the RMSE is 0.30 mmol/l. This difference in prediction error for specific subjects is due to the different amounts of noise present in different subjects' data. However, as can be seen from FIGS. 6-8, for a given subject, the models' performance is practically identical.

FIGS. 6 and 7 also reveal that sometimes a small time lag is introduced in the cross-subject and the cross-study scenarios. This small time lag is likely due to small differences in glucose dynamics across different individuals. AR models exhibit prediction lags if they failed to account for some frequency component present in the test signal. Such small differences in frequency components exist in the datasets and are the likely reason for the small prediction time lags. The introduction of a 5-minute lag for iSense subject #1 in scenario I (FIG. 6) is likely due to small frequency differences between this subject's training and testing data.

The results on model portability are valid for AR-type models. As discussed above, AR models capture the signal's frequency information and are invariant to the signal's phase and amplitude. The latter property is not shared by other modeling approaches, such as those based on ordinary differential equations or harmonic regression, which prevents their portability.

Accordingly, at least one embodiment of the invention develops stable, universal glucose models that capture the correlations in glucose time-series signals of diabetic patients. Given continuous glucose signals from a patient, such universal models are readily usable to make near-future glucose concentration predictions for other patients without any need for model customization.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The invention can take the form of an entirely hardware embodiment or an embodiment containing both hardware and software elements. In at least one exemplary embodiment, the invention is implemented in a processor (or other computing device) loaded with software, which includes but is not limited to firmware, resident software, microcode, etc.

Computer program code for carrying out operations of the present invention may be written in a variety of computer programming languages. The program code may be executed entirely on at least one computing device (or processor), as a stand-alone software package, or it may be executed partly on one computing device and partly on a remote computer. In the latter scenario, the remote computer may be connected directly to the one computing device via a LAN or a WAN (for example, Intranet), or the connection may be made indirectly through an external computer (for example, through the Internet, a secure network, a sneaker net, or some combination of these).

It will be understood that each block of the flowchart illustrations and block diagrams and combinations of those blocks can be implemented by computer program instructions and/or means. These computer program instructions may be provided to a processor of a general purpose computer, special purpose computer, application specific integrated circuit (ASIC), or other programmable data processing apparatus to produce a machine, such that the instructions, which execute via the processor of the computer or other programmable data processing apparatus, create means for implementing the functions specified in the flowcharts or block diagrams.

The invention has industrial applicability to predict future glucose levels in diabetic patients. The invention utilizes the predicted glucose levels to alter or improve the patient's lifestyle, to tighten their glycemic control, or to adjust therapy in a proactive manner. The universal AR models of the invention predict future glycemic states, which can be used to avoid undesired hypoglycemic or hyperglycemic episodes.

### REFERENCES

[1] D. C. Klonoff, “Continuous glucose monitoring—roadmap for 21st century diabetes therapy,” Diabetes Care, vol. 28, pp. 1231-1239, 2005.
[2] T. Bremer, D. A. Gough, “Is blood glucose predictable from previous values? A solicitation for data,” Diabetes, vol. 48, pp. 445-451, 1999.
[3] G. Sparacino, F. Zanderigo, S. Corazza, A. Maran, A. Facchinetti, and C. Cobelli, “Glucose concentration can be predicted ahead in time from continuous glucose monitoring sensor time-series,” IEEE Trans. Biomed. Eng., vol. 54, pp. 931-937, 2007.
[4] A. Gani, A. V. Gribok, S. Rajaraman, W. K. Ward, and J. Reifman, “Predicting Subcutaneous Glucose Concentration in Humans: Data-Driven Glucose Modeling,” IEEE Trans. Biomed. Eng., vol. 56, pp. 246-254, 2009.

1-15. (canceled)

16. A system for predicting at least one future glucose level of an individual, said system including:
a glucose measuring device, the glucose measuring device generates a series of glucose signals representing glucose levels obtained from the individual at fixed time intervals; and
an analyzer having a glucose prediction function that is portable between individuals irrespective of health of individuals, said glucose prediction function including a plurality of model coefficients that are invariant between individuals, said glucose prediction function outputs the at least one future glucose level by weighing the current and a plurality of previous series of glucose signals obtained from the individual by said model coefficients, said glucose prediction function outputs a series of future glucose levels by omitting the oldest predicted or actual glucose level used in the last iteration of said glucose prediction function, multiplying a most recent predicted future glucose level by a first model coefficient, and multiplying a next most recent predicted or actual glucose level by a next model coefficient.

17-27. (canceled)

28. A method, including:
receiving a time horizon as an input or retrieving the time horizon from memory;
receiving series of glucose signals from a glucose measuring device, the series of glucose signals representing glucose levels obtained from an individual at fixed time intervals;
predicting at least one future glucose level of the individual by weighing the series of glucose signals by a plurality of model coefficients of a glucose prediction function that is portable between individuals irrespective of health of individuals, said plurality of model coefficients are invariant between individuals, said weighing of the series of glucose signals by said plurality of model coefficients of said glucose prediction function includes omitting a least recent predicted or actual glucose level from said glucose prediction function, multiplying a most recent predicted future glucose level by a first model coefficient, and multiplying a next most recent predicted or actual glucose level by a next model coefficient, and said predicting being performed with a processor having code to perform calculations of said glucose prediction function; and
repeating said predicting for the number of required samples to reach the time horizon with each new prediction being one sampling time period later.

29. The method according to claim 28, wherein the health of the individual includes a diabetes type of the individual.

30. The method according to claim 28, wherein the health of the individual includes an age of the individual.

31. The method according to claim 30, wherein the health of the individual includes whether the individual is hospitalized.

32. The method according to claim 28, wherein said plurality of model coefficients are invariant between individuals irrespective of a type of said glucose measuring device utilized to measure the series of glucose signals.

33. The method according to claim 28, wherein said plurality of model coefficients number 30 and include a first coefficient having a value between 0.80 and 0.83, a second coefficient having a value between 0.50 and 0.52, a third coefficient having a value between 0.23 and 0.24, a fourth coefficient having a value between −0.01 and 0.02, a fifth coefficient having a value between −0.17 and −0.14, a sixth coefficient having a value between −0.25 and −0.23, a seventh coefficient having a value between −0.25 and −0.23, a eight coefficient having a value between −0.20 and −0.28, a ninth coefficient having a value between −0.12 and −0.11, a tenth coefficient having a value between −0.04 and −0.01, a eleventh coefficient having a value between 0.05 and 0.07, a twelveth coefficient having a value between 0.10 and 0.13, a thirteenth coefficient having a value between 0.13 and 0.15, a fourteenth coefficient having a value between 0.13 and 0.14, a fifteenth coefficient having a value between 0.10 and 0.11, a sixteenth coefficient having a value between 0.05 and 0.07, a seventeenth coefficient having a value between −0.01 and 0.01, a eighteenth coefficient having a value between −0.05 and −0.03, a nineteenth coefficient having a value between −0.08 and −0.06, a twentieth coefficient having a value between −0.09 and −0.07, a twenty-first coefficient having a value between −0.08 and −0.07, a twenty-second coefficient having a value between −0.06 and −0.05, a twenty-third coefficient having a value between −0.03 and −0.01, a twenty-fourth coefficient having a value between 0.00 and 0.02, a twenty-fifth coefficient having a value between 0.03 and 0.05, a twenty-sixth coefficient having a value between 0.04 and 0.06, a twenty-seventh coefficient having a value between 0.04 and 0.05, a twenty-eighth coefficient having a value between 0.02 and 0.03, a twenty-ninth coefficient having a value between −0.01 and 0.00, and a thirtieth coefficient having a value between −0.05 and −0.03.

34. The method according to claim 28, further including generating an alert when the at least one future glucose level of the individual at least one of exceeds an upper glucose threshold and falls below a lower glucose threshold.

35. The method according to claim 28, wherein said weighing of the series of glucose signals by said plurality of model coefficients reduces a time lag of the at least one future glucose level.

36. The method according to claim 28, further including displaying the at least one future glucose level on a display connected to said processor.

37. The method according to claim 28, further including storing the series of glucose signals in a memory.

38. The method according to claim 28, wherein said glucose prediction function is a universal autoregressive model.

39. The method according to claim 28, further including converting the series of glucose signals via said processor into numerical values representing the glucose levels obtained from the individual.

40-46. (canceled)

47. A method, including:
receiving series of glucose signals from a glucose measuring device, the series of glucose signals representing glucose levels obtained from an individual at fixed time intervals;
predicting at least one future glucose level of the individual by weighing the series of glucose signals by model coefficients of a glucose prediction function that is portable between individuals irrespective of diabetes types of individuals, ages of individuals, and type of said glucose measuring device,
said model coefficients are invariant between individuals; and
generating an alert when the at least one future glucose level of the individual is at least one of exceeding an upper glucose threshold and falling below a lower glucose threshold.

48. A method for predicting at least one future glucose level in an individual, said method including:
obtaining a plurality of first glucose measurements via a glucose monitoring device by monitoring current glucose levels at fixed time intervals in a plurality of individuals, said plurality of individuals having type I and type II diabetes;
training using a processor a glucose prediction function that is portable between individuals using at least a first portion of said plurality of first glucose measurements, said training including creating model coefficients that are invariant between individuals;
obtaining at least one second glucose measurement from the individual via one of said glucose monitoring device and a second glucose monitoring device; and
predicting the at least one future glucose level in the individual independent of whether the individual has type I or type II diabetes, said predicting including multiplying at least one of said model coefficients with at least one respective glucose measurement of said at least one second glucose measurement.

49. The method according to claim 48, wherein said training of said glucose prediction function and said predicting of the at least one future glucose level is independent of the type of glucose measurement device utilized to obtain said plurality of first glucose measurements and said at least one second glucose measurement.

50. The method according to claim 48, wherein said training of said glucose prediction function is independent of ages of said plurality of individuals, and wherein said predicting of the at least one future glucose level is independent of an age of the individual.

51. The method according to claim 50, wherein said training of said glucose prediction function is independent of whether said plurality of individuals are hospitalized, and wherein said predicting of the at least one future glucose level is independent of whether the individual is hospitalized.

52. The method according to claim 48, wherein said multiplying of said at least one of said model coefficients with said at least one respective glucose measurement reduces a time lag of the at least one future glucose level.

53. The method according to claim 48, wherein said predicting the at least one future glucose level includes predicting a future glucose level at least 5 minutes from said obtaining of said at least one second glucose measurement from the individual.

54. The method according to claim 48, wherein said glucose prediction function is a universal autoregressive model.

55-66. (canceled)
